(19) United States
(12) Patent Application Publication
(10) Pub. No.: US 2005/0272052 A1
(43) Pub. Date: Dec. 8, 2005

(54) Molecular genetic profiling of gleason grades 3 and 4/5 prostate cancer

(75) Inventors: Mamatha Shekar, Cupertino, CA (US); Zhaomei Zhang, Sunnyvale, CA (US); Mitchell C. Caldwell, Menlo Park, CA (US); Zuxiong Chen, Mountain View, CA (US); Zhenbin Fan, Mountain View, CA (US); John E. McNeal, Oakland, CA (US); Rosalie Nolley, Menlo Park, CA (US); Thomas A. Stamey, Portola Valley, CA (US); Janet A. Warrington, Los Altos, CA (US); John F. Palma, San Ramon, CA (US)

Correspondence Address: FISH & NEAVE IP GROUP;ROPES & GRAY, ONE INTERNATIONAL PLACE, BOSTON, MA, 02110, US

(73) Assignee: Affymetrix, INC., Santa Clara, CA (US)

(21) Appl. No.: 10/975,592

(22) Filed: Oct. 27, 2004

Related U.S. Application Data
(63) Continuation-in-part of application No. 10/411,537, filed on Apr. 9, 2003.
(60) Provisional application No. 60/371,304, filed on Apr. 9, 2002.

(51) Int. Cl. C12Q 1/68

(52) U.S. Cl. 435/6

(57) ABSTRACT

Many genes are affected in prostate cancers which have not been previously identified. This includes genes that have been up-regulated or down-regulated. Monitoring the expression levels of these genes is useful to identify the existence of prostate cancer. Also, monitoring the expression levels of these genes is useful to predict the effectiveness of treatment, outcome, use of therapeutics, and screening drugs useful for the treatment of prostate cancer.

### PRIORITY CLAIM

This application is a continuation-in-part of application Ser. No. 10/411,537 filed on Apr. 9, 2003, which is a non-provisional of Application No. 60/371,304 filed on Apr. 9, 2002, the disclosures of which are incorporated herein by reference for all purposes.

### RELATED APPLICATIONS

This application is also related to application Ser. No. 10/222,206, which is incorporated herein by reference for all purposes.

### FIELD OF THE INVENTION

The invention relates to the field of cancer diagnostics and therapeutics. In particular it relates to prostate cancer.

### BACKGROUND

Many cellular events and processes are characterized by altered expression levels of one or more genes. Differences in gene expression correlate with many physiological processes such as cell cycle progression, cell differentiation and cell death. Changes in gene expression patterns also correlate with changes in disease or pharmacological state. For example, the lack of sufficient expression of functional tumor suppressor genes and/or the over expression of oncogene/protooncogenes could lead to tumorgenesis (Marshall, Cell, 64: 313-326 (1991); Weinberg, Science, 254: 1138-1146 (1991), incorporated herein by reference for all purposes). Thus, changes in the expression levels of particular genes (e.g. oncogenes or tumor suppressors) serve as signposts for different physiological, pharmacological and disease states.

Prostate cancer, along with lung and colon cancer, are the three most common causes of death from cancer in men in the U.S., but prostate is by far the most prevalent of all human malignancies with the exception of skin cancer (Scott R. et al., J. Urol., 101:602,1969; Sakr W A et al., J. Urol., 150: 379, 1993). It is one of the top three causes of death from cancer in men in the United States (Greenlee R T et al., CA Cancer J. Clin. Vol 15, 2001) Currently, treatments available for prostate cancer require not only an early detection of the malignancy and a reliable assessment of the severity of the cancer.

The prostate is an heterogeneous gland, measuring 3-4 centimeters long by 3-5 centimeters in width, comprised of several concentric zones including central (CZ), peripheral (PZ) and transition (TZ) zones. PZ gives rise to about 80% of all prostate cancer, TZ gives rise to about 20% of cancer and BPH and prostate cancer is much less common in in the CZ (McNeal J E, Am. J. Clin. Path., 49:347, 1968)

Clinical and pathologic stage and histological grading system are being used to indicate prognosis fro group of patients based on the degree of tumor differentiation or the type of glandular pattern. A commonly used system for determining the prognosis of a patient with prostate cancer is the Gleason scoring system. The “Gleason score” or “Gleason grade” is a value from 1 (well differentiated) to 5 (poorly differentiated) based on the examination of slices of prostate cancer tissue under a microscope. The lower the Gleason score the more the prostate cancer tissue resembles the structure of normal prostate tissue and the less aggressive the cancer is likely to be.

The current primary diagnostic tool for prostate cancer detection is measurement of the level of prostate-specific antigen (PSA) in blood, which in normal men ranges from 0 to 4 nanograms/milliliters. Prostate enlargement, a condition known as benign prostatic hyperplasia (BPH), is found in half of the men over the age of 45. With BPH, PSA levels rise in proportion to prostate size, possibly obscuring diagnosis of cancer. In addition, a significant proportion of men with prostate cancer have normal PSA levels. Therefore the PSA test is somewhat non-specific to distinguish between BPH and prostate cancer. In the majority of the cases, PSA elevation is due to BPH rather than cancer.

Even though PSA levels has bee used as a marker for prostate cancer, it is largely related to BPH at PSA levels less than 12 ng/ml, poorly correlates with the volume of any Gleason grade cancers and does not correlates with any potential cure rates (Stamey T A et al., J. Urol, 167:103, 2002). Understanding the molecular meachanism of prostate cancer will help in identification of new prostate cancer serum markers and development or more accurate tests for correlating increasing grade 4/5 with curative outcome.

In previous studies, 9 histologic variables related to prostate cancer progression in 379 men were quantified with long-term follow-ups after radical prostatectomy using a detectable, rising prostate-specific antigen (PSA) as an indicator of progressive cancer. We found that the strongest histologic predictor of progression in radical prostatectomy specimens examined at 3-mm section intervals was the amount of Gleason grade 4/5 tumor in the largest peripheral zone (PZ) cancer. For every 10% increase in Gleason grade 4/5, we found a proportional 10% increase in post-radical prostatectomy PSA failure rates.

Although serum PSA between 2-12 ng/ml has been widely used in the U.S. as a potential marker for prostate cancer, in this range it is largely related to benign prostatic hyperplasia (BPH), a much more common disease. Moreover, we now know that serum PSA is poorly correlated with the volume of both high-grade (Gleason grade 4/5) and low-grade (Gleason grades 3, 2, and 1) prostate cancer, and that the level of pre-radical prostatectomy PSA does not discriminate between potential cure rates at PSA levels around 2-12 ng/ml. Adding to the PSA dilemma is our recent observation that preoperative positive prostatic biopsies have no dependable relationship to the important characteristics of the largest tumor within the prostate that determines cancer progression.

There is a need in the art for tumor markers for prostate cancer that can provide alternative measures to the notoriously inaccurate PSA. In particular, there is a need for markers for Gleason grade 4/5 prostate cancer, which is strongly related to poor outcome.

### SUMMARY OF THE INVENTION

According to one aspect of the invention a method is provided for predicting the outcome of cancer in a patient. The level of expression of at least one RNA transcript or its translation product in a first or a second group of RNA transcripts in a first sample of prostate tissue is compared to the level of expression of the transcripts or translation products in a second sample of prostate tissue. The first prostate tissue sample is neoplastic and the second prostate tissue sample is nonmalignant human prostate tissue. The first group of RNA transcripts consists of transcripts of genes selected from the group consisting of genes listed in FIGS. 9, 10, 11, 15, 16, 17, and the lower section of FIGS. 19, 20, 21 and 22 and the second group of RNA transcripts consists of transcripts of genes listed in FIGS. 6, 7, 8, 12, 13, 14 and the upper section of FIGS. 19, 20, 21 and 22. The patient is identified as having a poor outcome when expression of at least one of the first group of RNA transcripts or translation products is found to be lower in the first sample than in the second sample, or expression of at least one of the second group of transcripts or translation products is found to be higher in the first sample than in the second sample.

In another embodiment of the invention a method is provided for evaluating carcinogenicity of an agent to human prostate cells. The level of expression of at least one transcript or its translation product from a first or a second group of RNA transcripts is compared. The level of expression in a first sample of human prostate cells contacted with a test agent is compared to level of expression in a second sample of human prostate cells not contacted with the test agent. The first group of RNA transcripts consists of transcripts of genes selected from the group consisting of genes listed in FIG. 9, 10, 11, 15, 16, 17, and the lower section of FIGS. 19, 20, 21 and 22 and the second group of RNA transcripts consists of transcripts of genes listed in FIG. 6, 7, 8, 12, 13, 14 and the upper section of FIGS. 19, 20, 21 and 22. An agent is a potential carcinogen to human prostate cells if it decreases the level of expression of at least one of the genes of the first group, or increases the level of expression of at least one of the genes in the second group.

In another embodiment of the invention a method is provided for slowing progression of prostate cancer in a patient. A polynucleotide is administered to prostate cancer cells of the patient. The polynucleotide comprises a coding sequence of a gene selected from the group consisting of genes listed in FIG. 9, 10, 11, 15, 16, 17, and the lower section of FIGS. 19, 20, 21 and 22. The gene is expressed in the prostate cancer cells and slows progression of prostate cancer in the patient.

In another embodiment of the invention a method is provided for slowing progression of prostate cancer in a patient. An antisense construct is administered to prostate cancer cells of a patient. The antisense construct comprises at least 12 nucleotides of a coding sequence of a gene selected from the group consisting of gene listed in FIG. 6, 7, 8, 12, 13, 14 and the upper section of FIGS. 19, 20, 21 and 22. The coding sequence is in a 3′ to 5′ orientation with respect to a promoter that controls its expression, and an antisense RNA is expressed in cells of the cancer, slowing progression of prostate cancer in the patient.

In another embodiment of the invention a method is provided for slowing progression of prostate cancer in a patient. In this method an antibody is administered to prostate cancer cells in a patient. The antibody specifically binds to a protein expressed from a gene selected from the group consisting of genes in FIG. 6, 7, 8, 12, 13, 14 and the upper section of FIGS. 19, 20, 21 and 22. The antibody binds to the protein and slows progression of prostate cancer in the patient.

In another embodiment of the invention a method is provided for screening candidate drugs useful in the treatment of prostate cancer. A prostate cancer cell is contacted with a test substance. Expression of a transcript or translation product of a gene from a first or second group is monitored. The first group consists of genes listed in FIG. 9, 10, 11, 15, 16, 17, and the lower section of FIGS. 19, 20, 21 and 22 and the second group consists of genes listed in FIG. 6, 7, 8, 12, 13, 14 and the upper section of FIGS. 19, 20, 21 and 22. A test substance is identified as a potential drug useful for treating prostate cancer if it increases expression of at least one of the genes in the first group or decreases expression of at least one of the genes in the second group.

In another embodiment of the invention a method is provided for diagnosing prostate cancer in a patient. The level of expression of at least one RNA transcript or its translation product in a test sample of prostate tissue is compared to the level of expression of the at least one RNA transcript or translation product in a control sample of prostate tissue. The test sample of prostate tissue is suspected of being neoplastic and the control sample is nonmalignant human prostate tissue. At least one RNA transcript or its translation product is selected from a first or a second group of RNA transcripts or translation products. The first group of RNA transcripts consists of transcripts of genes selected from the group consisting of genes listed in FIG. 9, 10, 11, 15, 16, 17, and the lower section of FIGS. 19, 20, 21 and 22. The second group of RNA transcripts consists of transcripts of genes selected from the group consisting of genes listed in FIG. 6, 7, 8, 12, 13, 14 and the upper section of FIGS. 19, 20, 21 and 22. The test sample is identified as cancerous when expression of at least one of the first group of RNA transcripts or translation products is found to be lower in the test sample than in the control sample, or expression of at least one of the second group of transcripts or translation products is found to be higher in the test sample than in the control sample.

In another embodiment of the invention an array of nucleic acid molecules is provided. The nucleic acid molecules of the array comprise a set of members having distinct sequences, and each member is fixed at a distinct location on the array. At least 10% of the members on the array comprise at least 15 contiguous nucleotides of genes selected from the group consisting of genes in FIG. 9, 10, 11, 15, 16, 17, and the lower section of FIGS. 19, 20, 21 and 22, and genes listed in FIG. 6, 7, 8, 12, 13, 14 and the upper section of FIGS. 19, 20, 21 and 22.

In another embodiment of the invention a method is provided for monitoring or predicting the outcome of prostate cancer in a patient. The level of at least one serum marker is measured in a serum sample of a patient with prostate cancer. The serum marker is a protein expressed from a first or second group of genes. The first group of genes is selected from the group consisting of genes ranked 4, 7, 18, 22, 26, 30, 38, 41, 53, and 55 as shown in FIG. 6. The second group of genes consists of PLA2G7/LDL-phospholipase A2 (U24577).

The present inventions thus provide reagents and tools for diagnosing, slowing the progression of, and monitoring and predicting the outcome of prostate cancer in a patient. The present inventions also provide methods for evaluating carcinogenicity of an agent to human prostate cells, and for screening for candidate drugs for treating prostate cancer. Nucleic acid arrays are also provided.

According to one aspect of the invention a method is provided for predicting the outcome of cancer in a patient. Level of expression is compared of at least one RNA transcript or its translation product from a group of RNA transcripts in a first sample of prostate tissue to level of expression of the transcripts or translation products in a second sample of prostate tissue. The first prostate tissue sample is neoplastic and the second prostate tissue sample is nonmalignant human prostate tissue. The patient is identified as having a poor outcome when expression of at least one of the transcripts or translation products from the transcripts identified to be down-regulated in the G3 or G4/5 tissues identified is found to be lower in the first sample than in the second sample, or expression of at least one of transcripts or translation products from the transcripts identified to be up-regulated in G3 or G4/5 tissues is found to be higher in the first sample than in the second sample.

In another embodiment of the invention a method is provided for distinguishing between types of tumors.

G3 and G4/5 grade tumors were compared to either CZ or BPH samples to identify genes that are differentially expressed between the samples. When the G3 samples were compared to the CZ sample 23 transcripts were found to be up-regulated (FIG. 6) and 34 transcripts were found to be down-regulated (FIG. 9). When the G3 samples were compared to the BPH samples 10 were found to be up-regulated (FIG. 8) and 56 transcripts were found to be down regulated (FIG. 11). Transcripts were identified that were up-regulated (FIG. 7) and down-regulated (FIG. 10) in G3 when compared to both CZ and BPH.

In another embodiment of the invention a method is provided for distinguishing between normal, benign and malignant tissues.

In another embodiment of the invention a method is provided for diagnosing prostate cancer.

In another embodiment the invention provides serum markers for prostate cancer.

In another embodiment the invention provides potential drug targets for prostate cancer.

### BRIEF DESCRIPTION OF THE DRAWINGS

FIG. 1 shows a histogram of the relative expression levels measured using QRT-PCR and GeneChip microarray analyses for hepsin, maspin, single-minded homolog 2 and prostate differentiation factor from different samples. Sample 1-5 are G4/5 tumors, 6 and 7 CZ tissues, 8-11 are BPH. Hepsin is up-regulated in G4/5 cancer samples and Maspin is down-regulated in G4/5 cancer samples relative to CZ and BPH samples.

FIG. 2 shows the hierarchical clustering of samples with 20 genes identified by k-nearest neighbor clustering as having similar prediction accuracy than 1015 genes.

FIG. 3 is a table of the clinical and histological details of the 39 samples analyzed. The samples were one of four types: central zone (CZ), benign prostatic hyperplasia (BPH), Gleason grade 3 or 4/5 (G3 or G4/5) radical prostatectomy specimens.

FIG. 4 is a table with a summary of numbers of differentially expressed transcripts from six different comparisons.

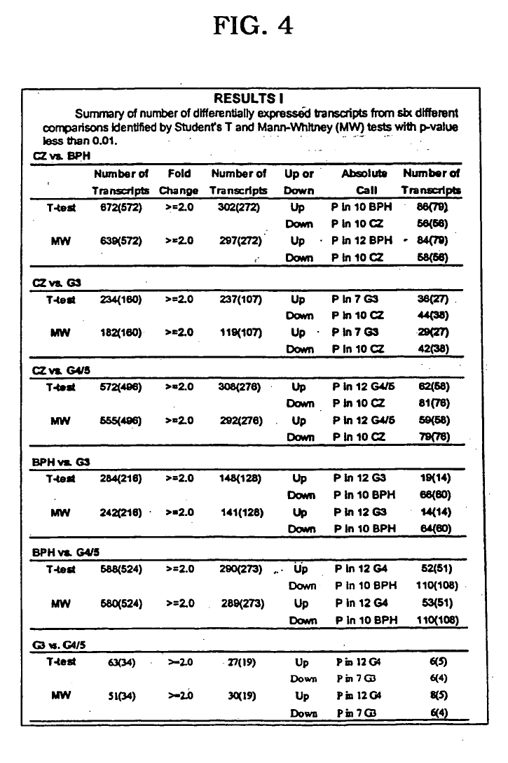

FIG. 5 is a table of a list of transcripts that are differentially expressed between CZ and BPH samples. The gene name, accession number and fold change are included.

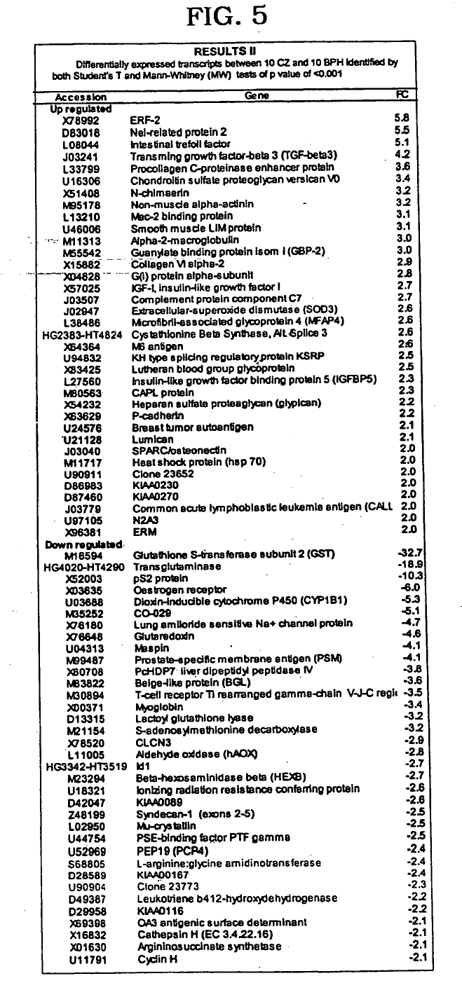

FIG. 6 is a table of transcripts that are up-regulated in G3 samples when compared to CZ samples but not when compared to BPH.

FIG. 7 is a table of transcripts that are up-regulated in G3 samples when compared to both CZ and BPH samples.

FIG. 8 is a table of transcripts that are upregulated in G3 samples when compared to BPH samples but not when compared to CZ samples.

FIG. 9 is a table of transcripts that are down-regulated in G3 samples when compared to CZ samples but not when compared to BPH samples.

FIG. 10 is table of transcripts that were found to be down-regulated in the G3 samples when compared to both the CZ and BPH samples.

FIG. 11 is a table of transcripts that were found to be down regulated in G3 samples compared to BPH samples but not when compared to CZ samples.

FIG. 12 is a table of transcripts that are up-regulated in G4/5 samples compared to CZ samples but not when compared to BPH samples.

FIG. 13 is a table of transcripts that are up-regulated in G4/5 sample when compared to both CZ and BPH samples.

FIG. 14 is a table of transcripts that are up regulated in G4/5 samples when compared to BPH samples but not when compared to CZ samples.

FIG. 15 is a table of transcripts that are down-regulated in G4/5 samples when compared to CZ samples but not when compared to BPH samples.

FIG. 16 is a table of transcripts that are down-regulated in G4/5 samples when compared to both CZ and BPH samples.

FIG. 17 is a table of transcripts that are down-regulated in G4/5 samples when compared to BPH samples but not when compared to CZ samples.

FIG. 18 is a table of transcripts that are differentially expressed between CZ and BPH samples.

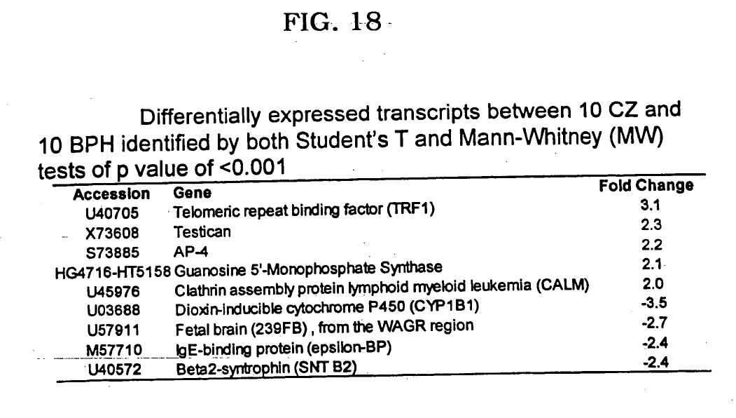

FIG. 19 is table of transcripts that are differentially expressed between CZ and G3 tumor using Human Genome U133A Affymetrix microarray.

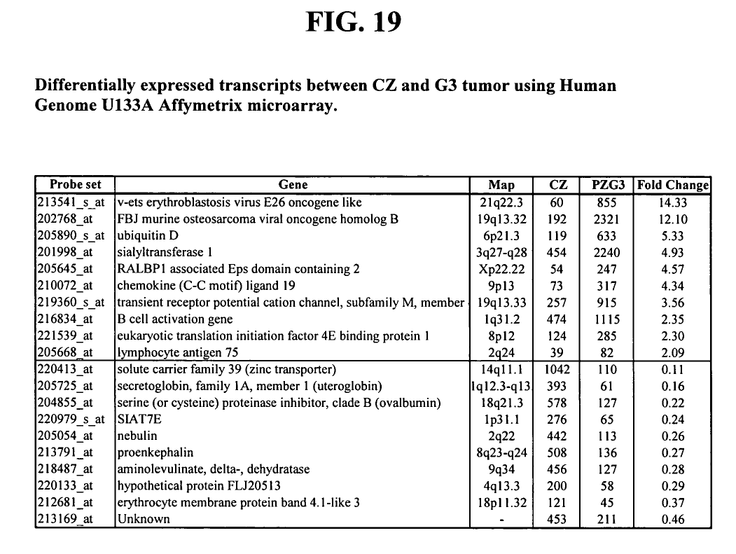

FIG. 20 is table of transcripts that are differentially expressed between CZ and G4/5 tumor using Human Genome U133A Affymetrix microarray.

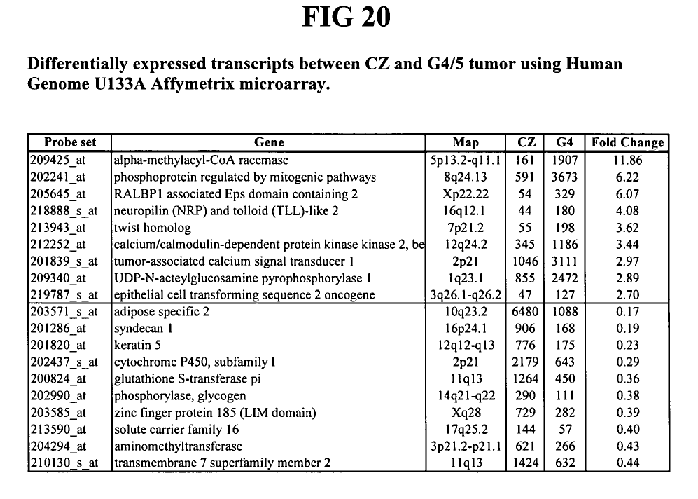

FIG. 21 is a table of transcripts that are differentially expressed between BPH and G3 tumor using Human Genome U133A Affymetrix microarray.

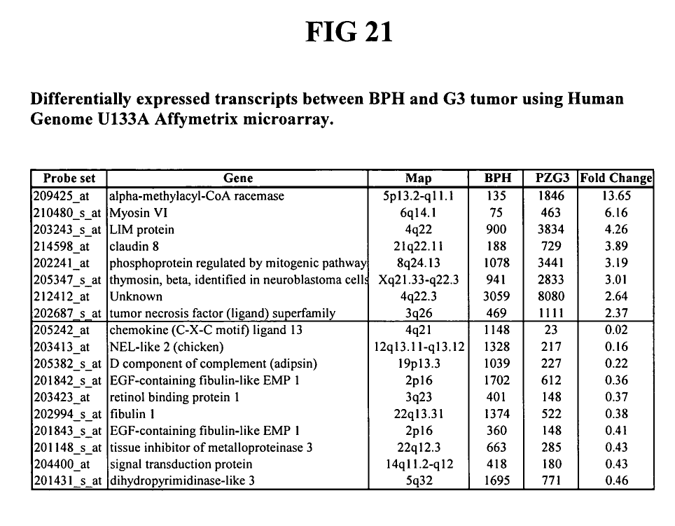

FIG. 22 is a table of transcripts that are differentially expressed between BPH and G4/5 tumor using Human Genome U133A Affymetrix microarray.

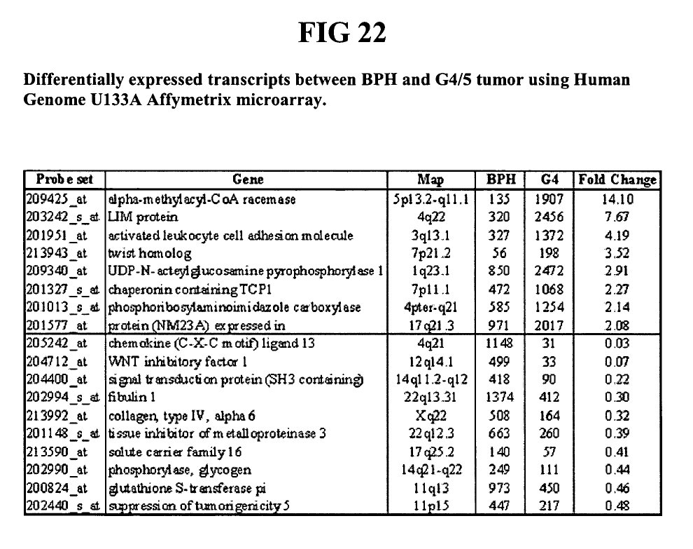

### DETAILED DESCRIPTION OF THE INVENTION

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes. The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

Arrays may be packaged in such a manner as to allow for diagnostics or can be an all-inclusive device; e.g., U.S. Pat. Nos. 5,856,174 and 5,922,591 incorporated in their entirety by reference for all purposes. (See also U.S. patent application Ser. No. 09/545,207 for additional information concerning arrays, their manufacture, and their characteristics.) It is hereby incorporated by reference in its entirety for all purposes.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248, 6,309,822 and 6,344,316. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

Those skilled in the art will recognize that the products and methods embodied in the present invention may be applied to a variety of systems, including commercially available gene expression monitoring systems involving nucleic acid probe arrays, membrane blots, microwells, beads, and sample tubes, constructed with various materials using various methods known in the art. Accordingly, the present invention is not limited to any particular environment, and the following description of specific embodiments of the present invention are for illustrative purposes only.

A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, nylon, polystyrene, polycarbonate, plastics, glass, ceramic, stainless steel, or the like. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded.

The gene expression monitoring system, in a preferred embodiment, may comprise a nucleic acid probe array (including an oligonucleotide array, a cDNA array, a spotted array, and the like), membrane blot (such as used in hybridization analysis such as Northern, Southern, dot, and the like), or microwells, sample tubes, beads or fibers (or any solid support comprising bound nucleic acids). See U.S. Pat. Nos. 5,770,722, 5,744,305, 5,677,195, 5,445,934, and 6,040,193 which are incorporated herein by reference. See also Examples, infra. The gene expression monitoring system may also comprise nucleic acid probes in solution.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617, 6,344,316 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. In a preferred embodiment, the proportional amplification methods of the present invention can provide reproducible results (i.e., within statistically significant margins of error or degrees of confidence) sufficient to facilitate the measurement of quantitative as well as qualitative differences in the tested samples. The proportional amplification methods of the present invention may also facilitate the identification of single nucleotide polymorphisms (SNPs) (i.e., point mutations that can serve, for example, as markers in the study of genetically inherited diseases) and other genotyping methods from limited sources. See, e.g., Francis S. Collins et al., Science 282:682 (1998). The mapping of SNPs can occur by any of various methods known in the art, one such method being described in U.S. Pat. No. 5,679,524, which is hereby incorporated by reference.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/AM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Definitions

“Nucleic acids” according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

“Nucleic acid library” or “array” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

“Combinatorial Synthesis Strategy”: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

“Complementary or substantially complementary”: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

“Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point™ fro the specific sequence at s defined ionic strength and pH. The Tm is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above.

“Hybridization probes” are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

“Hybridizing specifically to”: refers to the binding, duplexing, or hybridizing of a molecule substantially to or only to a particular nucleotide sequence or sequences under stringent conditions when that sequence is present in a complex mixture (e.g., total cellular) DNA or RNA.

“Probe”: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

“Target”: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

“mRNA or mRNA transcripts”: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A “fragment”, “segment”, or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

An “antibody” includes immunoglobulin molecules and immunologically active determinants of immunoglobulin molecules, i.e., molecules that contain an antigen binding site which specifically binds (immunoreacts with) an antigen. Structurally, the simplest naturally occurring antibody (e.g., IgG) comprises four polypeptide chains, two copies of a heavy (H) chain and two of a light (L) chain, all covalently linked by disulfide bonds. Specificity of binding in the large and diverse set of antibodies is found in the variable (V) determinant of the H and L chains; regions of the molecules that are primarily structural are constant (C) in this set. Antibody includes polyclonal antibodies, monoclonal antibodies, whole immunoglobulins, and antigen binding fragments of the immunoglobulins.

Microarray can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(supp.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high-density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hum. Mut. 7:244-255, both incorporated by reference in their entireties), the human immunodeficiency virus (HIV-1) reverse transcriptase and protease genes (U.S. Pat. No. 5,862,242 and Kozal et al., 1996, Extensive polymorphisms observed in HIV-1 clade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

The single-stranded or double-stranded DNA populations according to the present invention may refer to any mixture of two or more distinct species of single-stranded DNA or double-stranded DNA, which may include DNA representing genomic DNA, genes, gene fragments, oligonucleotides, PCR products, expressed sequence tags (ESTs), or nucleotide sequences corresponding to known or suspected single nucleotide polymorphisms (SNPs), having nucleotide sequences that may overlap in part or not at all when compared to one another. The species may be distinct based on any chemical or biological differences, including differences in base composition, order, length, or conformation. The single-stranded DNA population may be isolated or produced according to methods known in the art, and may include single-stranded cDNA produced from a mRNA template, single-stranded DNA isolated from double-stranded DNA, or single-stranded DNA synthesized as an oligonucleotide. The double-stranded DNA population may also be isolated according to methods known in the art, such as PCR, reverse transcription, and the like.

Where the nucleic acid sample contains RNA, the RNA may be total RNA, poly(A)⁺ RNA, mRNA, rRNA, or tRNA, and may be isolated according to methods known in the art. See, e.g, Sambrook and Russel, Molecular Cloning: A Laboratory Manual, (Cold Spring Harbor Lab., Cold Spring Harbor, N.Y. 2001). The RNA may be heterogeneous, referring to any mixture of two or more distinct species of RNA. The species may be distinct based on any chemical or biological differences, including differences in base composition, length, or conformation. The RNA may contain full length mRNAs or mRNA fragments (i.e., less than full length) resulting from in vivo, in situ, or in vitro transcriptional events involving corresponding genes, gene fragments, or other DNA templates. In a preferred embodiment, the mRNA population of the present invention may contain single-stranded poly(A)+ RNA, which may be obtained from a RNA mixture (e.g., a whole cell RNA preparation), for example, by affinity chromatography purification through an oligo-dT cellulose column.

Where the single-stranded DNA population of the present invention is cDNA produced from a mRNA population, it may be produced according to methods known in the art. See, e.g, Maniatis et al. In a preferred embodiment, a sample population of single-stranded poly(A)+ RNA may be used to produce corresponding cDNA in the presence of reverse transcriptase, oligo-dT primer(s) and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be thermal stable enzyme (e.g., hTth DNA polymerase).

Prostate cancer (PC), along with lung and colon cancer, are the three most common causes of death from cancer in men in the U.S. (See, Greenlee R T, et al. CA Cancer J Clin: 15, 200 which is incorporated herein by reference), but prostate is by far the most prevalent of all human malignancies with the exception of skin cancer (See, Scott R, et al. J Urol, 101: 602, 1969 and Sakr W A, et al. J Urol, 150: 379, 1993, which are incorporated herein by reference). In the United States, serum PSA of 2-12 ng/ml has been widely used as a potential marker for PC, but in this range it is largely related to benign prostatic hyperplasia (BPH), (See, Roehrbom C G, et al. J Urol, 163: 13, 2000, which is incorporated herein by reference), a much more common disease. Serum PSA poorly correlates with the volume of both high (4/5) and low (1-3) gleason grade cancers. Moreover, the level of pre-radical prostatectomy PSA between 2-12 ng/ml does not discriminate between potential cure rates (See, Stamey T A, et al. J Urol, January, 2002, which is incorporated herein by reference). Because grade 4/5 cancer is the primary cause of failure to cure prostate cancer, gene expression characterization of grade 3 and 4/5 cancers may help in the identification of new PC serum markers and the development of more accurate tests for correlating increasing grade 4/5 cancer with curative outcome (See, Stamey T A, et al. JAMA, 281: 1395, 1999, which is incorporated herein by reference).

III. Methods

Labeled targets from 9-10 central zone (CZ), 10 BPH, 13 PZ, 7 G3 and 12-16 G4/5 tissues were hybridized to high-density DNA microarrays containing probes representing ˜6800 full-length human genes. In addition to using BPH as the control normal samples to look for differential gene expression patterns in G3 and G4/5 cancers, CZ was also used as a control as it is virtually resistant to the development of PC (See, McNeal, J. E. Am J Clin Path, 49: 347, 1968, which is incorporated herein by reference). Using a number of analysis methods including Student's T-test, Mann-Whitney test, and hierarchical clustering, a number of genes exhibiting profound expression differences were identified that distinguish the tissue types. Hepsin and maspin were up and down regulated respectively in tumor tissues compared to both BPH and CZ. Depending on whether BPH or CZ tissue was used as the reference baseline, distinct sets of genes differentially expressed in tumor tissues were identified.

The results showed that expression profiles can distinguish BPH from G3 and G4/5 tumors and identified candidate genes to be used.

Hierarchical clustering of samples was done using the expression profile of 359 genes and each of the 39 samples accurately segregated into normal, benign and malignant tissues using genes differentially expressed between CZ, BPH, G3, G4/5. Identifying 359 candidate genes that provide molecular information for the development of improved diagnostics and new treatment choices.

A specific differential pattern of gene expression between benign prostate hyperplasia (BPH) and Gleason 3 and 4/5 carcinoma has been discovered. The differentially expressed genes may be used to diagnose prostate carcinoma, predict the outcome of prostate carcinoma, and slow the progression of prostate cancer. Prostate carcinoma may be diagnosed, or the outcome of prostate carcinoma may be predicted, by comparing levels of RNA transcripts or translation products, or comparing levels of serum markers between samples. Administering antibodies, antisense, or genes of the invention may slow the progression of prostate cancer. The differentially expressed genes may also be used to evaluate the carcinogenicity of an agent to human prostate cells, to screen for drugs to treat prostate carcinoma, and on nucleic acid arrays.

Many methods of the invention compare level of expression of RNA transcripts or translation products. Measuring the level of expression of these RNA transcripts or translation products may be performed by any means known in the art. Examples of methods to determine protein levels include immunochemistry such as radioimmunoassay, Western blotting, and immunohistochemistry. RNA levels may be measured using an array of oligonucleotide probes immobilized on a solid support. Northern blotting and in situ hybridization may also be performed to determine levels of RNA transcripts in samples. Comparison can be done by observation, by calculation, by optical detectors, or by computers, or any other means.

The levels of expression of these RNA transcripts or translation products are compared in methods of the invention, for instance, between different samples of prostate tissue. Higher levels of expression are defined as any statistically significant increase in expression of the RNA transcripts or translation products from one prostate sample relative to another prostate sample. The increase in expression may be, for example, 1.5-, 2-, 3-, 4.0-, 5-, or 10-fold higher, or more. Lower levels of expression are defined as any statistically significant decrease in expression of the RNA transcripts or translation products from one prostate sample relative to another prostate sample. The decrease in expression may be, for example, 1.5-, 2-, 3-, 4.0-, 5-, or 10-fold lower or more.

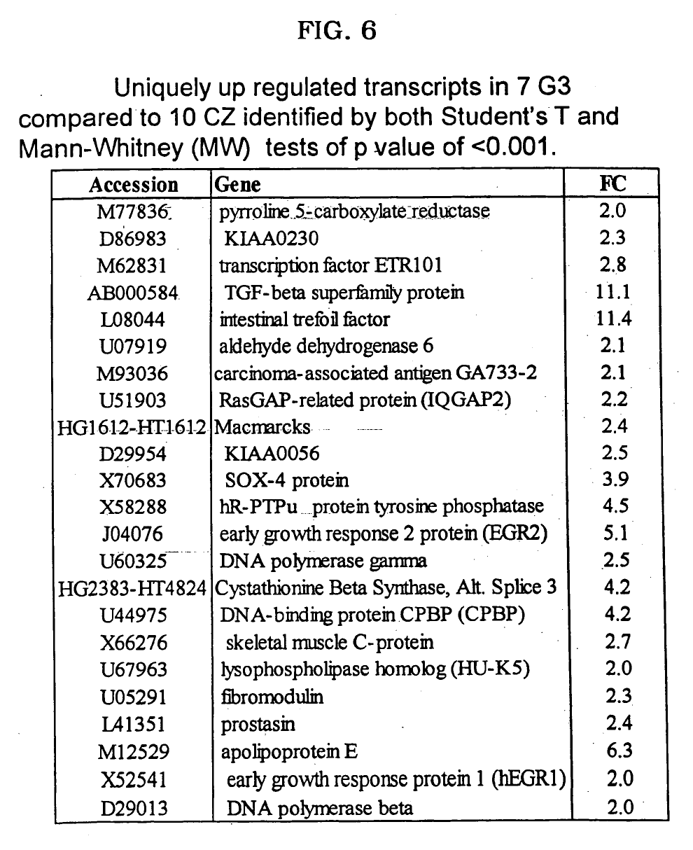

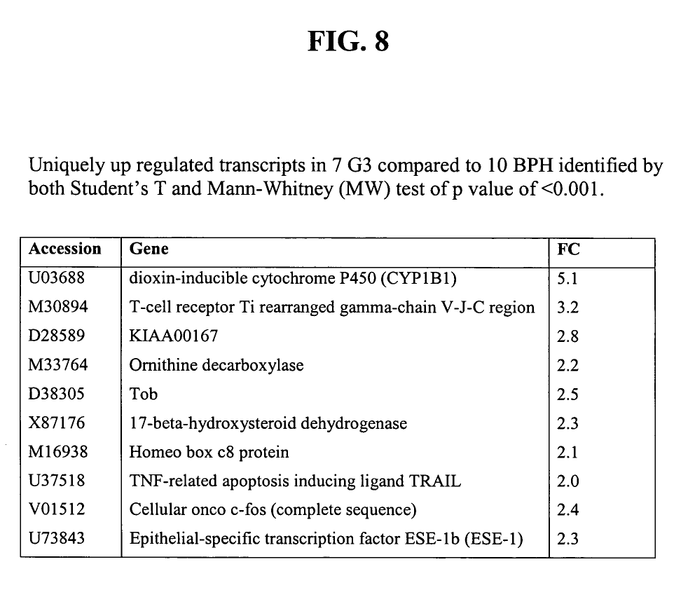

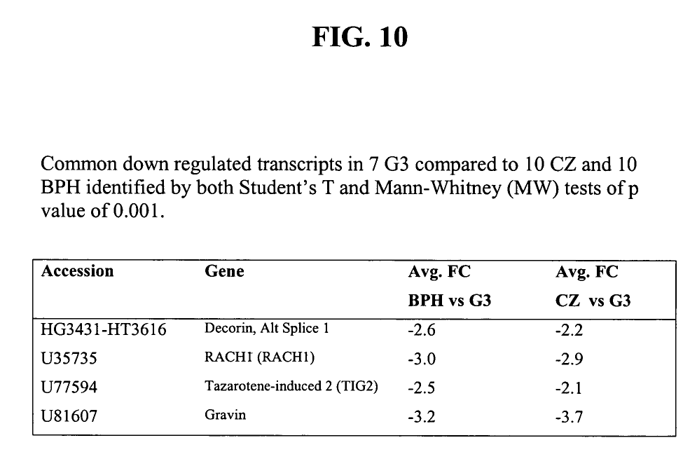

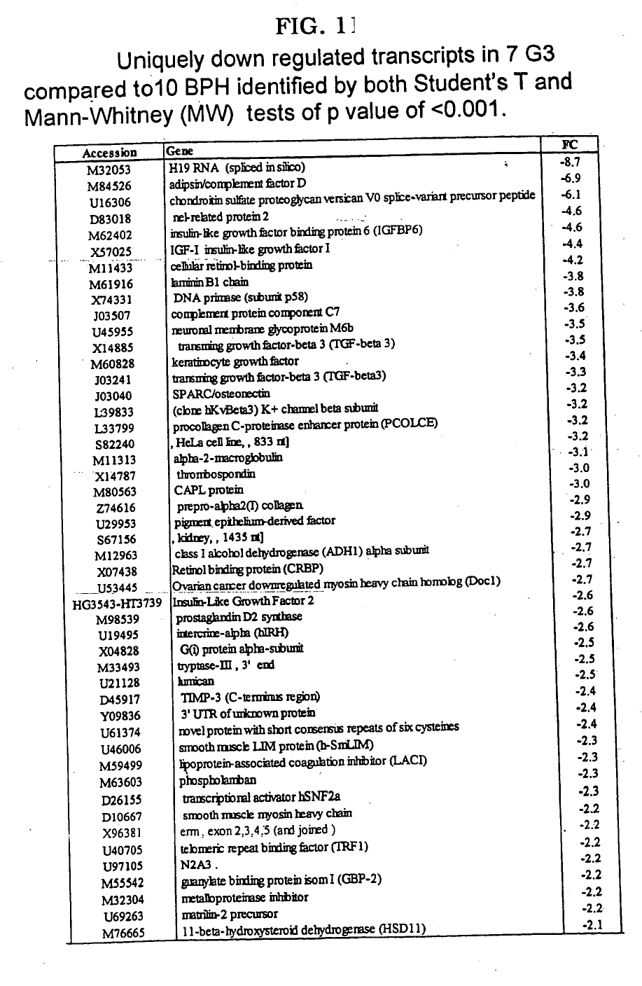

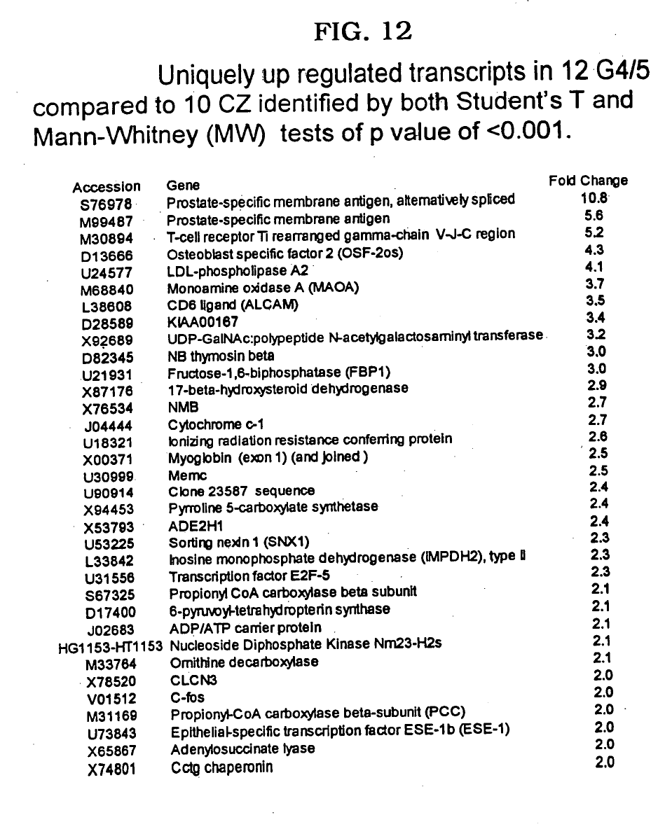

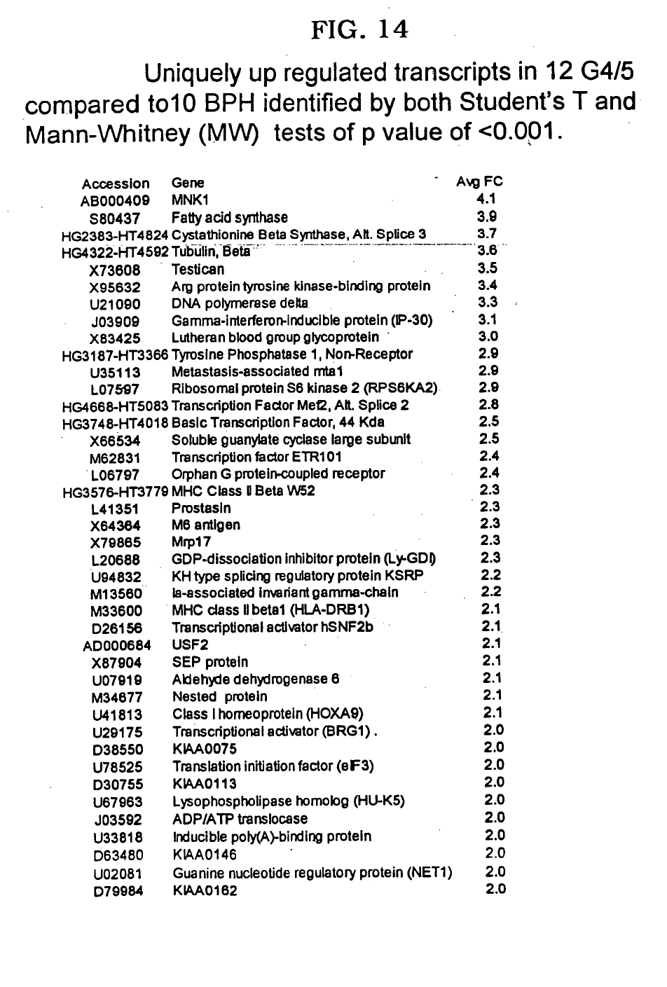

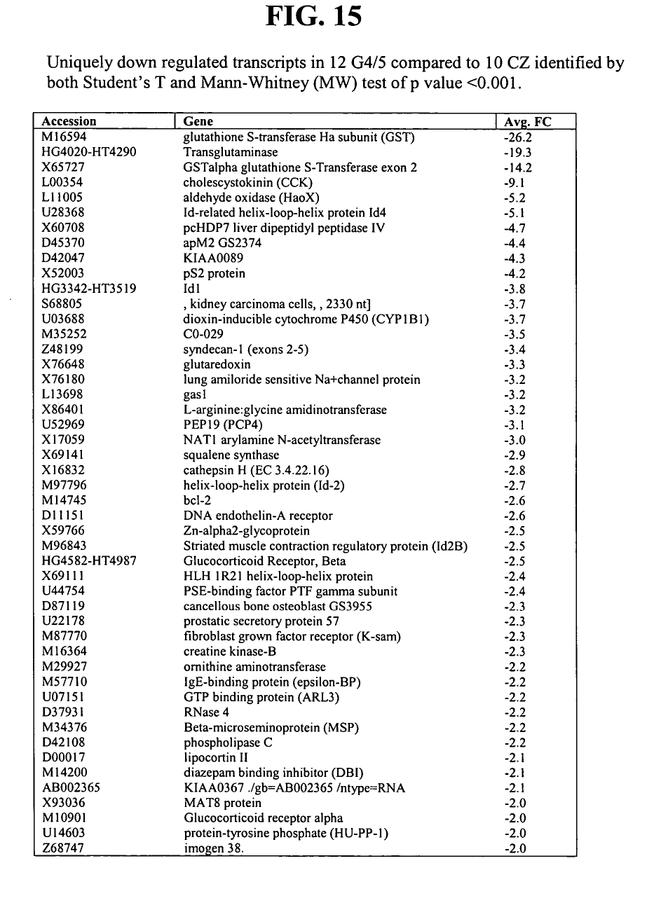

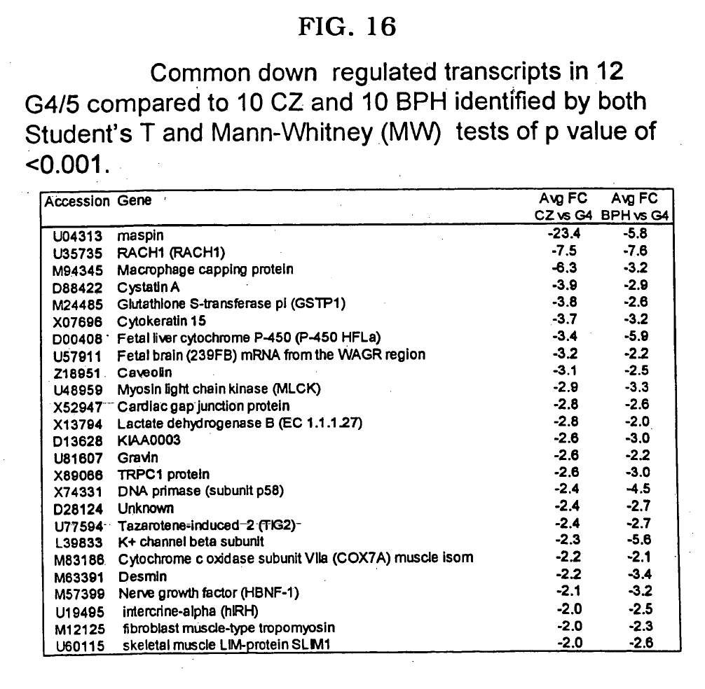

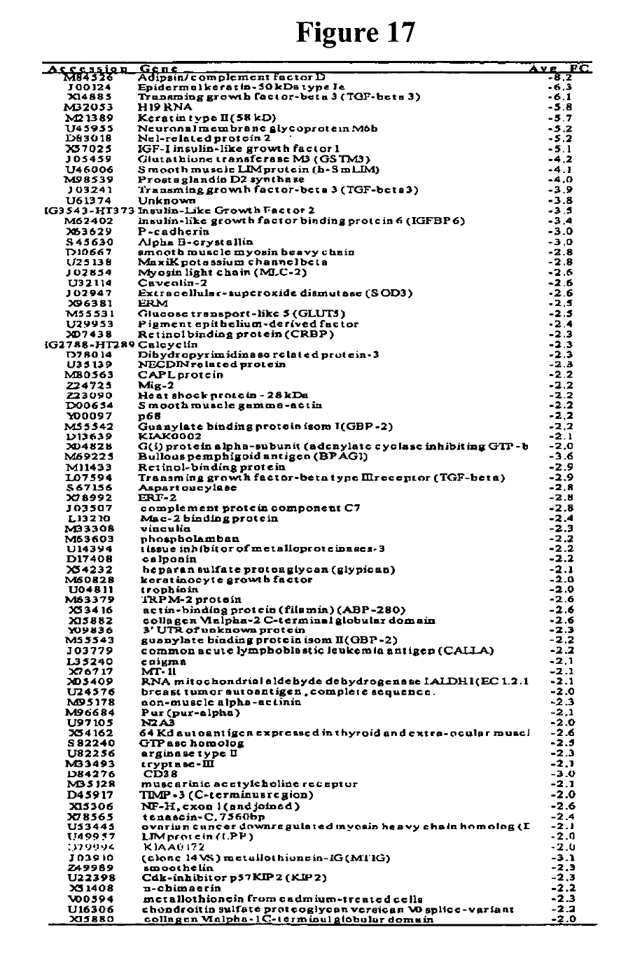

The outcome of prostate cancer in a patient can be predicted. Level of expression is compared of at least one RNA transcript or its translation product, in a first sample of prostate tissue that is neoplastic to a second sample of human prostate tissue that is nonmalignant. The transcript is a transcript of a gene selected from the genes listed in FIGS. 6-17.

Neoplastic prostate tissue exhibits abnormal histology that is consistent with cancerous cell growth at any stage of disease. The neoplastic tissue may be characterized as any of Gleason grades 1, 2, 3, 4, or 5. Neoplastic cells of Gleason grade 4/5 are particularly useful. Nonmalignant prostate tissue is free of any pathologically detectable cancer. The nonmalignant prostate tissue may be free of any prostate disease or abnormal growth. The nonmalignant tissue may also be benign prostate hyperplasia tissue.

A poor outcome is the result of progression of the neoplastic tissue from one Gleason grade to a higher Gleason grade. A poor outcome is associated with Gleason 4/5 prostate cancer. Even no change in marker pattern from a prior measurement may be characterized as a poor outcome.

Transcripts or translation products may be compared of at least 2, 5, 10, 20, 30, or 49 of the genes identified in the study. The information supplied by the groups of genes identified may provide increased confidence in the findings. Transcripts from the different groups may be compared.

Transcripts that are differentially regulated in G3 or in G4/G5 when compared to both CZ and BPH may be particularly useful for outcome prediction.

Carcinogenicity of an agent to human prostate cells can be evaluated using the genes involved in prostate cancer. Level of expression is compared of at least one transcript or its translation product from an identified RNA transcripts. A first sample of human prostate cells is contacted with a test agent and a second sample of human prostate cells is not contacted with the test agent. The levels of expression of at least 1, 2, 5, 10, 20, 50, 60, or 69 of the RNA transcripts or translation products may be compared. An agent is identified as a potential carcinogen to human prostate cells if it decreases the level of expression of at least one of the genes of the first group, or increases the level of expression of at least one of the genes in the second group.

Test agents may include any compound either associated or not previously associated with carcinogenesis of any cell type. Nonlimiting examples of test agents include chemical compounds that mutagenize DNA, or environmental factors such as ultraviolet light. Test agents also include pesticides, ionizing radiation, cigarette smoke, and other agents known in the art. Test agents may also be proteins normally found in the human body that cause abnormal changes in prostate cells or environmental factors known to induce tumors in other human tissues but that have not yet been associated with prostate cancer.

Any level of changed expression that may be induced in prostate cells identifies carcinogenicity. Desirably the change in expression is statistically significant and includes a change of at least 50%, 200%, 300%, 400%, or 500%.

Nonmalignant human prostate cells may be isolated from any human prostate free of malignant disease. The human prostate cells may also be human prostate cells that have been maintained in culture, such as transformed cell lines, that are nonmalignant. Nonmalignant includes both disease free and benign prostate hyperplasia.

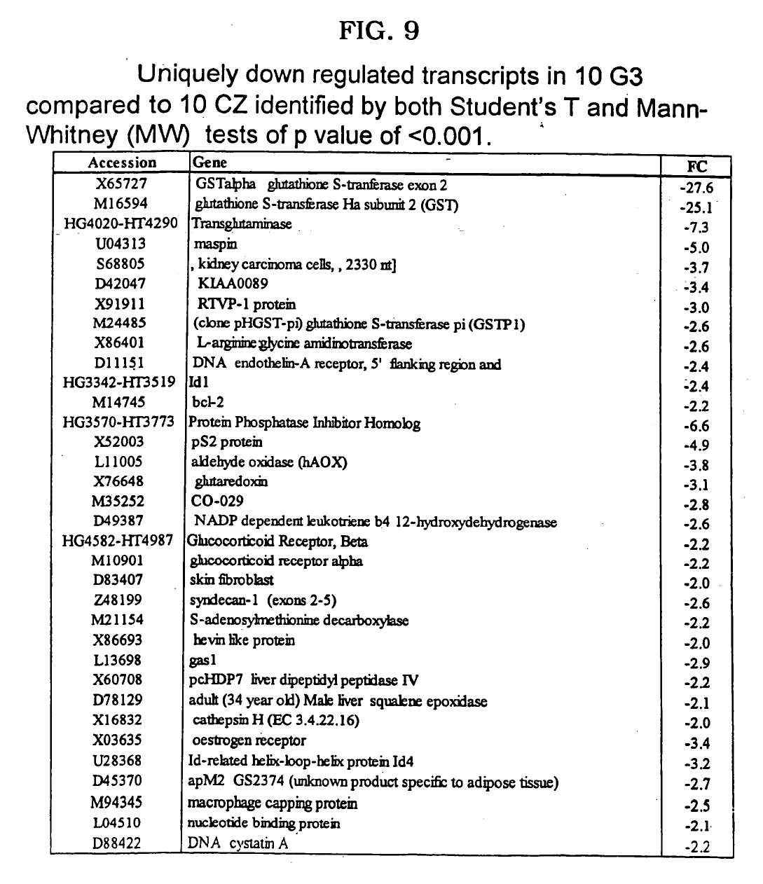

In order to slow progression of prostate cancer in a patient one can administer to the patient a polynucleotide comprising a coding sequence of a gene that is down-regulated in cancerous tissue in relation to nonmalignant tissue, for example the transcripts of FIGS. 9-11 and 15-17. Administration of the gene slows progression of prostate cancer in the patient.

An antisense construct can be administered to prostate cells of a patient. The antisense construct comprises at least 12 nucleotides of a coding sequence of a gene selected from the genes shown in FIGS. 6-8 and 12-14. The coding sequence is in a 3′ to 5′ orientation with respect to a promoter which controls its expression, whereby an antisense RNA is expressed in cells of the cancer and progression of prostate cancer in the patient is slowed. Alternatively, antisense oligonucleotides that bind to mRNA can be directly administered without a vector.

An antibody that specifically binds to a protein expressed from a gene selected from the genes shown in FIGS. 6-8 or 12-14 can be administered to a patient. The antibody binds to the protein and progression of prostate cancer is slowed in the patient.

Slowing progression of prostate cancer in a patient includes reduction of the rate of growth of prostate tumors at the prostate of the patient. Slowing progression of prostate cancer in a patient also includes a reduction in the rate of spread of the prostate tumor from the prostate to other sites in a patient. Furthermore, slowing progression of prostate cancer includes a reduction in the size of the prostate tumor, or the prevention of the spread of the prostate cancer in the patient. Any amount or type of reduced progression of the prostate cancer is desirable.

A polynucleotide includes all or a portion of the coding sequence of any of the genes identified. The gene segment may be linear, cloned into a plasmid, cloned into a human artificial chromosome, or cloned into another vector. Vectors also include viruses that are used for gene delivery. Viruses include herpes simplex virus, adenovirus, adeno-associated virus, or a retrovirus. The adenoviral vector may be helper virus dependent. The naked DNA may also be injected, or may be associated with lipid preparations, such as liposomes.

Any nucleic acid that binds to the identified genes or the RNA transcripts of the identified genes and prevents expression of their products can be used as a therapeutic antisense reagent. The antisense may be an oligonucleotide or ribozyme, or any other such polynucleotide known in the art. The antisense RNA will bind anywhere along the identified genes or RNA transcripts, including within the coding region or regulatory region of the gene sequence. The antisense also does not have to be perfectly complementary to the sequence of the identified genes or transcripts. It may also be of any effective length. The antisense polynucleotide may be at least 12, 15, 18, 21, 24, 27, 28, 29, or 30 bases in length. The antisense may or may not be driven by a promoter.

A promoter is a sequence that drives expression of RNA. Any of the suitable promoters known in the art may be used. The promoter may be a strong promoter derived from a virus, such as the mouse mammary tumor virus promoter, or Rous sarcoma virus promoter. The promoter may also be constitutive promoter that is active in all tissues, or may be a tissue specific promoter. Preferably, a tissue-specific promoter is a promoter specific to the prostate. Several nonlimiting examples of such promoters are the prostate specific antigen (PSA) promoter, the probasin (PB) promoter, and the prostate specific membrane antigen promoter.

Any modifications, such as the introduction of phosphorothioate bonds in the polynucleotides, may be made to increase the half-life of antisense polynucleotides in the patient. Other non-phosphodiester internucleotide linkages that may be introduced into the polynucleotides include phosphorodithioate, alkylphosphonate, alkylphosphonothioate, alkylphosphonate, phosphoramidate, phosphate ester, carbamate, acetamidate, carboxymethyl esters, carbonates, and phosphate triester. The bases or sugars of the nucleotides may be modified as well. For instance, arabinose may be substituted for ribose in the antisense oligonucleotide.

Administration of the gene or antisense construct can be by any acceptable means in the art. These include injection of the nucleic acids systemically into the bloodstream of the patient or into the prostate tumor directly. The nucleotides may also be administered topically or orally. The gene or antisense construct may be formulated with an excipient such as a carbohydrate or protein filler, starch, cellulose, gums, or proteins such as gelatin and collagen. The gene or antisense construct may be formulated in an aqueous solution. Preferably the solution is in a physiologically compatible buffer. Acceptable buffers include Hanks' solution, Ringer's solution, or physiologically buffered saline.

Antibodies that specifically bind to any epitope of the indicated proteins will slow the progression of the prostate cancer. The antibodies may be of any isotype, for example, IgM, IgD, IgG, IgE, or IgA. The antibodies may be full-length or may be a fragment or derivative thereof. For instance, the antibodies may be only the single chain variable domain, or fragments of the single chain variable domain. The antibodies may be in a monoclonal or a polyclonal preparation. The antibodies may also be produced from any source and may be conjugated to toxins or other foreign moieties. The antibodies may be produced using the hybridoma technique or the human B-cell hybridoma technique. They may also be produced by injection of peptide into animals such as guinea pigs, rabbits, or mice. Antibodies preferably bind to serum markers or cell surface proteins. The antibodies can be humanized or chimeric.

Candidate drugs can be screened for those useful in the treatment of prostate cancer. Prostate cancer cells can be contacted with a test substance. Expression of a transcript from FIGS. 6-17 or its translation product from a first or second group is monitored. A test substance is identified as a candidate drug useful for treating prostate cancer if it increases expression of at least one of the genes in the first group or decreases expression of at least one of the genes in the second group.

A test substance can be a pharmacologic agent already known in the art for another purpose, or an agent that has not yet been identified for any pharmacologic purpose. It may be a naturally occurring molecule or a molecule developed through combinatorial chemistry or using rational drug design. A test substance also may be nucleic acid molecules or proteins. These may or may not be found in nature.

Test substances are identified as candidate drugs if they increase expression of at least one of the genes that is down-regulated in G3 or G4/5 or decrease expression of at least one of the genes that is up-regulated in G3 or G4/5. Candidate drugs, as used herein, are drugs that are potentially useful for treating cancer. It is contemplated that further tests may be needed to evaluate their clinical potential after identification in the method. Such tests include animal models and toxicity testing, inter alia.

Prostate cancer can be diagnosed by comparing the level of expression of at least one RNA transcript or its translation product from the differentially expressed genes identified in FIGS. 6-17. The test sample is identified as cancerous when expression of at least one of the first group of RNA transcript or translation products is found to be lower in the test sample than in the control sample, or expression of at least one of the second group of transcripts or translation products is found to be higher in the test sample than in the control sample. Any number of transcripts can be compared.

For example, the level of expression of at least 1, 2, 5, 10, 20, 30, or 49 transcripts of the up-regulated group may be compared. Alternatively, the level of expression of at least 1, 2, 5, 10, or 20 transcripts of the down-regulated group may be compared. Alternatively, at least 2, 5, 10, or 20 transcripts of each of the up regulated and down regulated groups are compared. Alternatively, at least 30 transcripts or translation products in the up regulated group and 20 transcripts or translation products in the down regulated group, or 40 transcripts or translation products in the up regulated group and 20 transcripts or translation products in the down regulated group, or 49 transcripts or translation products in the up regulated group and 20 transcripts or translation products in the down regulated group are compared. The at least one transcript or translation product of the down-regulated group preferably comprises the transcript of the gene maspin. The at least one RNA transcript or its translation product of the up-regulated group of RNA transcripts preferably includes hepsin.

Arrays of nucleic acids comprise nucleic acid molecules which have distinct sequences that are fixed at distinct locations on the array. The GeneChip® system (Affymetrix, Santa Clara, Calif.) is a particularly suitable array, however, it will be apparent to those of skill in the art that any similar systems or other effectively equivalent detection methods can also be used. Nucleotide arrays are disclosed in U.S. Pat. Nos. 5,510,270, 5,744,305, 5,837,832, and 6,197,506, each of which is incorporated by reference. The nucleotide array is typically made up of a support on which probes are arranged. The support may be a chip, slide, beads, glass, or any other substrate known in the art. Oligonucleotide probes are immobilized on the solid support for analysis of the target sequence or sequences. For methods of attaching a molecule with a reactive site to a support see U.S. Pat. No. 6,022,963. For probes that may be used with arrays see U.S. Pat. No. 6,156,501. For methods of monitoring expression with arrays see U.S. Pat. Nos. 5,925,525 and 6,040,138, all of which are incorporated herein by reference.

The specific embodiments described above do not limit the scope of the present invention in any way as they are single illustrations of individual aspects of the invention. Functionally equivalent methods and components are within the scope of the invention. The scope of the appended claims thus includes modifications that will become apparent to those skilled in the art from the foregoing description.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

### IV. EXAMPLES

### Example 1

### Characterization of the Upregulated and Downregulated Genes Specifically in Gleason Grade 3 Cancer and Gleason Grade 4/5 Cancers Using BPH or/and CZ as a Control for Increased or Decreased Expression

Labeled targets (cRNAs) from 10 central zone (CZ), 10 BPH, 7 G3 and 12 G4/5 tissues were hybridized to high-density DNA microarrays containing probes representing ˜6800 full-length human genes. Nodules of BPH were used as control for several reasons, the most important of which is the histologic heterogeneous nature of the prostate. Other reasons for using nodules of BPH as control cells for gene expression analysis include the histologic identity of PZ epithelial cells and TZ epithelial cells when viewed with the high power of the microscope although they are readily distinguishable with the low-power field by the incorporation of TZ cells into a pattern of nodular architecture. More importantly, it is observed that almost all available antibodies for studying prostate epithelium appear to stain both PZ and TZ epithelial types equivalently. Finally, a complete transverse section across the mid-gland of any prostate>50 grams in size is almost certain to reveal some nodules of BPH. While “normal” PZ cells would be ideal as control epithelium for PZ grade 4/5 cancer, unfortunately epithelial atrophy and dysplasia, the latter of which gives rise to Gleason grade 3 cancer in the PZ, are very common in prostates from men>50 years old. McNeal, J. E., Villers, A., Redwine, E. A., Freiha, F. S., Stamey, T. A., Microcarcinoma in the prostate: Its association with duct-acinar dysplasia. Human Pathology, 22:644-652, 1991, herein incorporated by reference in its entirety. For these reasons, the gene transcripts from Gleason grade 4/5 cancer were compared to nodules of BPH.

In addition to using BPH as the control normal samples to look for differential gene expression patterns in G3 and G4/5 cancers, CZ was also used as a control as it is virtually resistant to the development of PC (See, McNeal, J. E. Am J Clin Path, 49: 347, 1968, which is incorporated herein by reference).

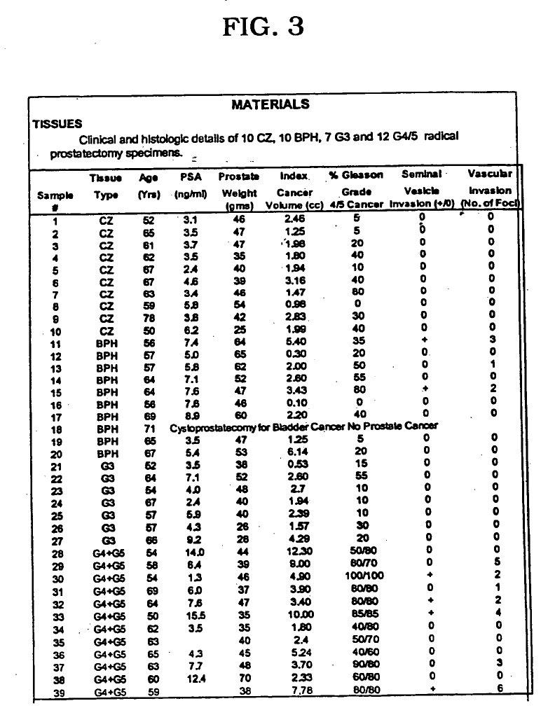

Samples of prostatic tissue were obtained within 15 minutes of intraoperative interruption of the blood supply to the prostate. Patient age, preoperative serum PSA levels, and histologic details of the 17 prostates are provided in FIG. 3 for the radical prostatectomy specimens submitted for RNA extraction.

Trimmed prostate tissue blocks or the ten 60-micron sections were homogenized with trizol reagent using a power homogenizer (Polytron) for 10 minutes and incubated at room temperature for five minutes to allow complete dissociation of nucleobinding proteins. To the homogenized samples, 0.2 ml was added, of chloroform per 1.0 ml of trizol reagent, which was vigorously shaken by hand for 15 seconds and incubated at room temperature for three minutes. The samples were then centrifuged at 12,000×g for 15 minutes in a cold room; 0.6 ml of the colorless upper aqueous phase (that contained tissue total RNA) was transferred to a fresh tube. Isopropyl alcohol (0.5 ml) and 1 μl of glycogen were used to precipitate RNA at room temperature. After 15 minutes, the RNA pellets were obtained by centrifugation at 12K×g for 10 minutes in a cold room, washed twice with 75% ethanol by vortexing, followed by centrifugation. Total RNA was further purified using the RNeasy® Mini Kit (Qiagen, Inc., Valencia, Calif., USA) according to the manufacturer's instructions.

Double-strand cDNA was synthesized from total RNA; labeled cRNA was prepared from cDNA, as described by Mahadevappa and Warrmgton and applied to HuGeneFl® probe arrays representing ≈6,800 genes or human genome U133A Affymetrix GeneChip® array containing ≈22,000 genes. Mahadevappa, M., Warrington, J. A., A high density probe array sample preparation method using 10-100 fold fewer cells. Nature Biotech, 17:1134-1136, 1999, herein incorporated by reference in its entirety. The arrays were synthesized using light-directed combinatorial chemistry, as described by Fodor et al. Fodor, S. P. A., Read, J. L., Pirrung, M. C., Stryer, L., Lu, A. T., Solas, D., Light-directed spatially addressable parallel chemical synthesis, Science, 251: 713-844, 1991 and Fodor, S. P. A., Rava, R. P., Huang, X. C., Pease, A. C., Holmes, C. P., and Adams, C. L., Multiplexed biochemical assays with biological chips, Science, 364: 555-556, 1993, which are herein incorporated by reference in their entirety.

All procedures were carried out as described by Warrington et al. Warrington, J. A., Nair, A., Mahadevappa, M., Tsyganskaya, M., Comparison of human adult and fetal expression and identification of 535 housekeeping/maintenance genes, Phys Genomics, 2: 143-147, 2000, herein incorporated by reference in its entirety.

Sample quality was assessed by agarose gel electrophoresis and spectrophotometry (A260/A280 ratio) using aliquots of total RNA to evaluate whether or not the RNA was of sufficient quality to continue. If the total RNA appeared intact, the samples were prepared and hybridized to the GeneChip® Test3 Array (Affymetrix, Inc., Santa Clara, Calif.) to determine the ratio of 3′ and 5′ GAPDH (glyceraldehydes 3-phosphate dehydrogenase) transcript levels and finally to the HuGeneFl arrays human genome U133A Affymetrix GeneChip® array.

Labeled cRNA was applied to HuGeneFL® probe arrays representing ≈6,800 genes or to the human genome U133A Affymetrix GeneChip® array representing ≈22,000 genes and processed according to Affymetrix protocols. Datasets are prepared by Affymetrix Microarray Suite® Version 4.0.1, filtered and sorted by Microsoft® Excell 2002, and statistically analyzed by S-PLUS® and Insightful Miner 2.0. the results are confirmed by Significance Analysis of Microarrays (SAM; Tusher V G et al. P.N.A.S. , Vol 98:5116, 2001).

Depending on whether BPH or CZ tissue was used as the reference baseline, distinct sets of genes differentially expressed in tumor tissues were identified.

### Example 2

### Data Analysis and Data Reduction

The primary purpose of data analysis in gene array experiments is data reduction. To accomplish this, several software tools were used for data analysis, including Microsoft Access and Microsoft Excel (Redmond, Wash. 98052-6399) and Affymetrix Microarray Suite (Santa Clara, Calif. 95051). Microarrarray Suite was used to analyze the sacn image with default parameter settings and all experiment were scaled to target intensity of 300. The ˜6,800 human genes represented on the HuGeneFL® probe array or the ˜22,000 human genes represented on U133A are comprised of probes of single-stranded DNA oligonucleotides 25 bases long, designed to be complementary to a specific sequence of genetic information. Hundreds of thousands to millions of copies of each probe inhabit a probe cell and each cell is a member of a probe pair. Half of that probe pair is comprised of cells that contain exact copies of the DNA sequence, a “Perfect Match”; the companion cell in the probe pair contains copies of the sequence that are altered only at the 13th base, a “Mismatch,” which serves as a control for the Perfect Match sequences. There are 16-20 probe pairs per probe set and each probe set represents one gene. The probe sets are measured for fluorescence, which is proportional to the degree of hybridization between the labeled cRNA from our tissue sample and the DNA on the chip. An average of the differences in fluorescence between the Perfect Match and Mismatch pairs is calculated; this “Average Difference” value is critical and is used in all subsequent calculations for up and down regulation of each gene. Several other values are calculated, one of which, an assessment of whether mRNAs are present, absent, or marginal (“Absolute Call”, is used in other calculations). Warrington, J., Dee, S., Trulson, M., Large-scale genomic analysis using Affymetrix GeneChip® probe arrays. In: Microarray Biochip Technology. Edited by M. Schena, Naick, Mass.: Easton Publishing; chapter 6, 119-148, 2000, herein incorporated by reference in its entirety. All probe sets that were undetectable in all nine cancers and eight BPH samples were removed and the data set with descriptive statistics was examined.

Statistical analysis and subsequent ranking were carried out using Student t-test (unpaired, two-tailed, equal variance) and Mann-Withney test in GeneSpring (Silicon Genetics) Only up and down regulated genes with a p-value difference in fluorescence between grade 3 or 4/5 cancer and control (BPH or CZ) of p<0.0001 were selected. Additionally, two-dimensional and multidimensional clustering patterns were carried out using GenExplore (Applied Maths, Kortrijk, Belgium) and MATLAB (MathWorks, Natick, Mass.). A threshold was applied that eliminated all genes that were not increased or decreased by at least 2 times (2 fold change) in a comparison of every one of the BPH and/or CZ and grade 3 or 4/5 tissues and ranked them in terms of up and down regulation. This selection was confirmed by using the recently published technique of Tusher, Tibshirani and Chu. Tusher, V. G., Tibshirani, R., and Chu, G., Significance analysis of microarrays applied to the ionizing radiation response, Proc Natl Acad Sci USA, 98: 51165121, 2001, herein incorporated by reference in its entirety.

Hierarchical clustering of samples was done using the expression profile of 359 genes and each of the 39 samples accurately segregated into normal, benign and malignant tissues using genes differentially expressed between CZ, BPH, G3, G4/5. Identifying 359 candidate genes that provide molecular information for the development of improved diagnostics and new treatment choices.

### Example 3

### Class Prediction

Twenty genes from 1015 candidates genes were selected by k-nearest neighbor method (GeneSpring) for class prediction using 75% of the samples from each class as training set and remaining 25% as test set.

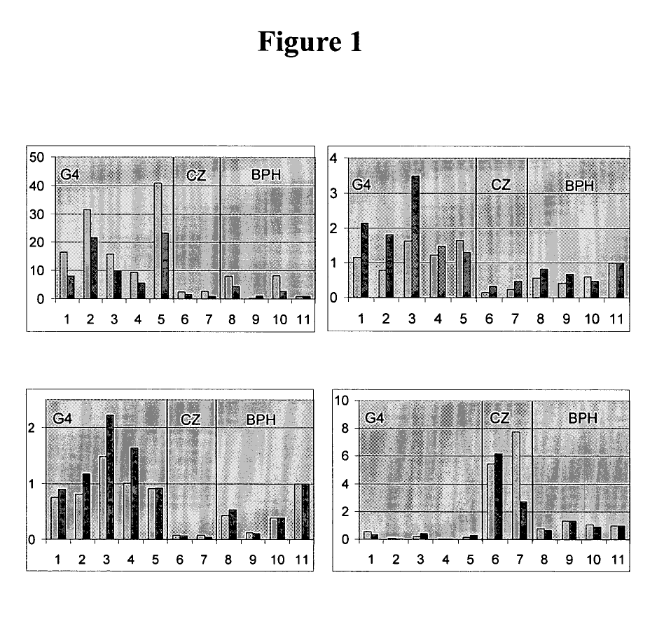

FIG. 1 shows that hierarchical clustering of samples with 20 genes identified by k-nearest neighbor clustering as having similar prediction accuracy as that of 1015 genes.

### Example 4

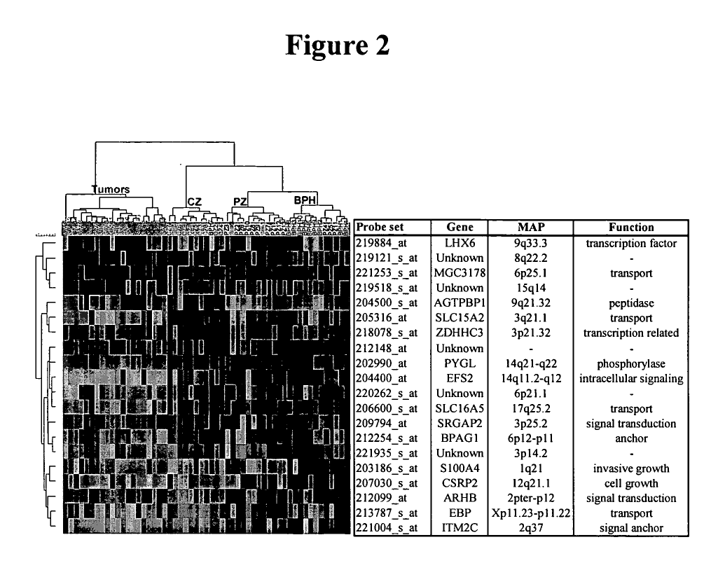

### Hepsin and Maspin were Up and Down Regulated Respectively in Tumor Tissues Compared to BPH or/and CZ (FIG. 2) as Shown by Microarray and by QRT-PCR

Four up and down regulated genes were selected for confirmation of microarray results based on statistical significance, fold change and biological relevance in the comparison of grade 4/5 cancers with normal CZ and BPH samples. Subsets of the original tissues used for the microarray analysis were selected for quantitative real time PCR analysis (Bieche I et al., Cli. Chem., 45:1148, 1999). QRT-PCR were performed according to Applied Biosystems' instructions. QRT-PCR results confirmed the array-based expression results.

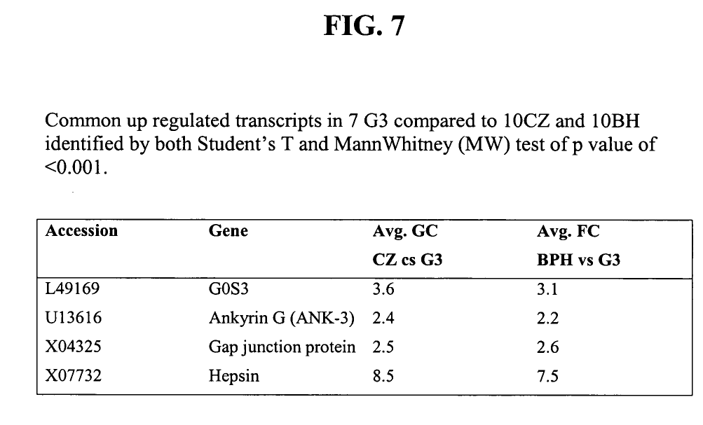

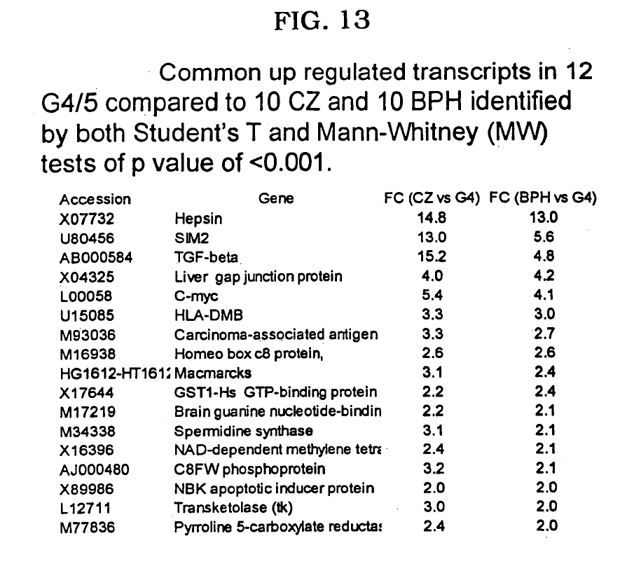

Of the most up regulated genes, hepsin is obviously of key interest (FIG. 7 and FIG. 13). It has been most intensely investigated in the cardiovascular field. Wit, Q., Yu, D., Post, J., Halks-Miller, M., Sadler, J. E., Morser, J., Generation and characterization of mice deficient in hepsin, a hepatic transmembrane serine protease, J Clin Invest, 101: 321-326, 1998, herein incorporated by reference in its entirety. It is known to be overexpressed in ovarian cancer. Tanimoto, H., Yan, Y., Clarke, J., Hepsin, a cell surface serine protease identified in hepatoma cells, is overexpressed in ovarian cancer. Cancer Res, 57:2884, 1997, herein incorporated by reference in its entirety. Hepsin is a type II cell surface trypsin-like serine protease with its enzyme's catalytic domain oriented extracellularly.

It is interesting that maspin, a serine protease inhibitor is the most down regulated gene; i.e., maspin is 23 times more expressed in CZ than in grade 4/5 cancer, potentially supporting, rather than inhibiting, the protease activity of hepsin in Gleason grade 4/5 cancer.

Prostate-specific membrane antigen (PSMA), the second most over-expressed gene, is present in prostate tissue and, importantly, in nonprostatic tumor neovasculature. Chang, S. S., O'Keefe, D. S., Bacich, D. J., Reuter, V. E., Heston, W. D. W., Gaudm, P. B., Prostate-specific membrane antigen is produced in tumor-associated neovasculature, Clin Cancer Res, 5: 2674-2681, 1999, herein incorporated by reference in its entirety. All earlier reports have been at the protein level. Our paper is the first report that the PSMA gene is highly over-expressed in the prostate and specifically in Gleason grade 4/5 cancer, which may broaden its potential therapeutic applications in the treatment of prostate cancer. In one immunohistochemical study, antibodies to PSMA stained Gleason grade 4/5 cells more intensely than grades 3, 2, and 1. Darson, M. F., Pacelli, A., Roche, P., et al, Human glandular kallikrem 2 (hK2) expression in prostatic intraepithelial neoplasia and adenocarcinoma: a novel prostate cancer marker. Urol, 49:857, 1997, herein incorporated by reference in its entirety.

The results showed that expression profiles can distinguish BPH and/or CZ from G3 and G4/5 tumors and identified candidate genes to be used.

1. A method for diagnosing prostate cancer in a patient, comprising the steps of: comparing level of expression of at least one RNA transcript or its translation product in a test sample of prostate tissue to level of expression of the at least one transcript or translation product in a control sample of prostate tissue, wherein the test sample of prostate tissue is suspected of being neoplastic and the control sample is nonmalignant prostate tissue, wherein the at least one RNA transcript or its translation product is selected from a first or a second group of RNA transcripts or translation products, wherein the first group of RNA transcripts consists of transcripts of genes selected from the group consisting of GST alpha glutathione S-transferase exon 2 (X65727), Glutathione S-transferase Ha subunit 2 (GST) (M16594), transglutaminase (HG4020-HT4290), P15-protease inhibitor 5 (maspin) (U04313), L Arg:Gly amidinotransferase (S68805), KIAA0089 (D42047), RTVP-1 protein (X91911), GSTP1 (glutathione S-transferase pi) (M24485), L-arginine:glycine amidinotransferase (X86401), DNA endothelin-A receptor (D11151), Id1 (HG3342-HT3519), bcl-2 (M14745), Protein Phosphatase Inhibitor Homolog (HG3570-HG3773), pS2 protein (X52003), HAOX (aldehyde oxidase) (L11005), glutaredoxin (X76648), CO-029 (M35252), NADP dependent leukotriene b412-hydroxydehydrogenase (D49387), Glucocorticoid receptor alpha (M10901), Glucocorticoid receptor beta (HG4582-HT4987), ZAKI-4 from skin fibroblast (D83407), syndecan (exon 2-5) (Z48199), S-adenosylmethionine decarboxylase (M21154), hevin-like protein (X86693), gas 1 (L13698), pcHDP7 liver dipeptidyl peptidase IV (X60708), adult male liver squalene epoxidase (D78129), cathepsin H (X16832), oestrogen receptor (X03635), Id-related helix-loop-helix protein Id4 (U28368), apM2 GS2374 (D45370), macrophage capping protein (M94345), nucleotide binding protein (L04510), DNA cystatin A (D88422), Decorin (HG3431-HT3616), RACH1 (U35735), TIG2 (tazarotene-induced 2) (U77594), gravin (U81607), H19 RNA (M32053), adipsin/complement factor D (M84526), chondoitin sulfate proteoglycan versican V0splice-variant precursor peptide (U16306), nel-related protein 2 (D83018), IGFBP6 (insulin-like growth factor I) (X57025), cellular retinol-binding protein (M11433), laminin B1 chain (M61916), DNA primase (subunit 58) (X74331), complement protein component C7 (J03507), neuronal membrane glycoprotein M6b (U45955), TGF-beta3 (transming growth factor-beta3) (X14885), keratonocyte growth factor (M60828), SPARC/osteonectin (J03040), K+ channel beta subunit (L39833), procollagen C-proteinase enhancer protein (PCOLCE) (L33799), GTPase homolog HeLA cell line 833 nt (S82240), alpha-2 macroglobulin (M11313), thrombospondin (X14787), CAPL protein (M80563), prepro-alpha2(I) collagen (Z74616), pigment epithelium-derived factor (U29953), aspartoacylase kidney 1435 nt (S67156), class I alcohol dehydrogenase (ADH1) alpha subunit (M12963), CRBP (retinol binding protein) (X07438), Ovarian cancer down-regulated myosin heavy chain homolog (Doc1) (U53445), Insulin-like Growth factor 2 (HG3543-HT3739), Prostaglandin D2 synthase (M98539), hIRH (intecrin-alpha) (U19495), G9i) protein alpha-subunit (X04828), tryptase-III 3′-end (M33403), lumican (U21128), TIMP-3: C-terminus region (D45917), 3′UTR of unknown protein (Y09836), novel protein with short consensus repeats of six cysteines (U61374), h-SmLIM (smooth muscle LIM protein) (U46006), LACI (lipoprotein-associated coagulation inhibitor) (M59499), phospholamban (M63603), transcriptional activator hSNF2a (D26155), smooth muscle myosin heavy chain (D10667), erm exon2,3,4,5 (X96381), telomeric repeat binding factor (TRF1), N2A3 (U97105), GBP-2 (guanylate binding protein isom I) (M55542), metalloproteinase inhibitor (M32304), matrilin-2 precursor, 11-HSD11 (beta-hydroxysteroid dehydrogenase) (M76665), CCK (cholecystokinin) (L00354), apM2 GS2374 (D42047), CYP1B1 (dioxin-inducible cytochrome P450) (U03688), lung amiloride sensitive Na+ channel protein (X76180), PCP4 (PEP19) (U52969), NAT1 (anylamine N-acetyltransferase) (X17059), squalene synthase (X69141), Id-2 (helix-loop-helix protein) (M97796), Zn-alpha2-glycoprotein (X59766), Striated muscle contraction regulatory protein (Id2B) (M96843), Glucocorticoid receptor Beta (HG4582-HT4087), HLH 12R1 helix-loop-helix protein (X69111), PSE-binding factor PTF gamma subunit (U44754), cancellous bone osteoblast GS3955 (D87119), prostatic secretory protein 57 (U22178), K-sa, (Fibroblast Growth Factor Receptor) (M87770), creatine kinase-B (M16364), ornithine aminotrasnferase (M29927), epsilon-BP (IgE-binding protein) (M57710), ARL3 (GTP binding protein) (U07151), RNase 4 (D37921), MSP (Beta-microsemiprotein) (M34376), phospholipase C (D42108), lipocortin II (D00017), DBI (diazepam binding inhibitor) (M14200), KIAA0367 (AB002365), MAT8 protein (X93036), protein-tyrosin phosphatase (HU-PP-1) (U14603), imogen38 (Z68747), Cystatin A (D88422), Cytokeratine 15 (X07696), P-450 HFLa (Fetal liver cytochrome P-450) (D00408), Fetal brain (239FB) mRNA from the WAGR region (U57911), Caveolin (Z18951), MLCK (myosin light chain kinase) (U48959), cardiac gap junction protein (X52947), lactate dehydrogenase B (Ec 1.1.1.27) (X13794), KIA0003 (D13628), TRPC1 protein (X89066), unknown protein (D28124), K⁺ channel beta subunit (L39833), COX7A (cytochrome c oxidase subunit VIIa muscle isoforms (M83186), desmin (M63391), HBNF-1 (nerve growth factor) (M57399), hIRH intercrien-alpha (U19495), fibroblast muscle-type tropomyosin (M12125), SLIM1 (skeletal muscle LIM-protein) (U60115), Adipsin/complement factor D (M84526), Epidermalkeratin-50 kDa type Ie (J00124), H-19 RNA (M32053), Keratin type II 58 kD (M21389), neuronal membrane glycoprotein M6B (U45955), GS TM3 (Glutathione transferase M3) (J05459), unknown protein (U61374), Insulin-like growth factor-2 (IG3543-HT373), IGFBP6 (insulin-like growth factor binding protein 6) (M62402), P-cadherin (X63629), alpha-B crystalline (S45630), MaxiK potassium channel beta (U25138), MLC-2 (myosin light chain) (J02854), caveolin 2 (U32114), SOD3 (extracellular superoxide dismutase) (J02947), ERM (X96381), GLUT5 (Glucose transport-like 5) (M55531), pigment epithelium derived factor (U29953), CRBP (retinol binding protein) (X07438), calcyclin (IG2788-HT289), dehydropyrimidinase related protein-3 (D78014), NECDIN related protein (U35139), CAPL protein (M80563), Mig-2 (Z24725), Heat shock protein 28 kDa (Z23090), smooth muscle gamma-actin (D00654), p68 (Y00097), KIAK002 (D13639), G9i) protein-alpha subunit (adelynate cyclase inhibiting GTP-b) (X04828), BPAG1 (Bullous pemphigoid antigen) (M69225), retinol-binding protein (M11433), TGF beta (transming growth factor-beta type III receptor) ((L07594), aspartoacylase (S67156), ERF-2 (X78992), complement protein component C7 (J03507), Mac-2 binding protein (L13210), vinculin (M33308), phospholamban (M63603), tissue inhibitor of metalloproteinase 3 (U14394), calponin ((D17408), glypican (hepara sulfate proteoglycan (X54232), keratinocyte growth factor (M60828), trophinin (U04811), TRPM-2 protein (M63379), filamin ABP-280 (actin binding protein) ((X53416), collagen VI alpha 2C-terminal globular domain (X15882), GBP-2 (guanylate binding protein II) (M55543), CALLA (common acute lymphoblastic leukemia antigen) (J03779), enigma ((L35240), MT-11 (X76717), ALDHI (RNA mitochondrial aldehyde dehydrogenase) (X05409), breat tumor antigen (U24576), non-muscle alpha-actinin (M95178), pur (pur-alpha) (M96684), N2A3 (U97105), 64 kD autoantigen expressed in thyroid and extra-occular muscle (X54162), GTPase homolog (S82240), arginase type II (U82256), tryptase-III (M33493), CD3 8 (D84276), muscarinic acetylcholine receptor (M35128), NF-H exon 1 (X15306), tenascin-C 7560 bp (X78565), LPP (IIM protein) (U49957), KIA0172 (D79994), MTIG (clone 14 VS metallothionein-IG) (J03910), smoothelin (Z49989), KIP 2 (Cdk-inhibitor p57 KIP1 (U22398), n-chimaerin (X51408), metallothionein from cadmium-treated cells (V00594), collagen VI alpha-1C-terminal globular domain (X15880), soluble carrier family 39 (zinc transporter) (NM_{—}014579.1), secretoglobin family 1A member I (uteroglobin) (NM_{—}003357.1), serine or cystrein proteinase inhibitor (NM_{—}002639.1), SIAT7E (NM_{—}030965), nebulin (NM_{—}004543.2), proenkephalin (NM_{—}006211.1), aminolevulinate delta dehydratase (BC000977.1), hypothetical protein FIJ20513 (NM_{—}017855.1), erythrocyte membrane protein band 4.1-like 3 (AI770004), adipose specific 2, unknown protein (BG109855), syndecan 1 (Z48199), keratin 5 (NM_{—}000424.1), cytochrome p450 subfamily 1 (NM_{—}000104.2), glutathione S-transferase pi (NM_{—}000852.2), phosphorylase glycogen (NM_{—}002863.1), zinc finger protein 185 (LIM domain) (NM_{—}007150.1), single carrier family 16 AA705628), aminoethyltransferase (NM_{—}000481), transmembrane 7 superfamily member 2 (AF096304.1), chemokine (C-X-C motif) ligand 13 (NM_{—}006419.1), NEL-like 2 (NM_{—}006159.1), D component of complement (adipsin) (NM_{—}001928.1), EGF-containing fibulin-like EMP-1 (AI826799), retinol binding protein 1 (NM_{—}002899.2), fibulin 1 (Z95331), tissue inhibitor of metalloproteinase 3 (NM_{—}000362.2), signal transduction protein (NM_{—}005864.1), dihydrpyramidinase-like 3 (NM_{—}001387.1), WNT inhibitory factor 1 (NM_{—}007191.1), signal transduction protein (SH3 containing)(NM_{—}005864.1), collagen type IV alpha 6 (AI889941), suppression of tumorigenicity 5 (NM_{—}005418.1),
and wherein the second group of RNA transcripts or translation products are being selected from the group consisting of pyrroline 5-carboxylase reductase (M77836), KIAA0230 (D86983), transcription factor ETR10 (M62831), TGF-beta superfamily (AB000584), intestinal trefoil factor (L08044), aldehyde dehydrogenase 6 (U07919), carcinoma associated antigen GA733-2 (M93036), IQGAP2 (RasGAP-related protein) (U51903), Macmarcks (HG1612-HT1612), KIAA0056 (D29954), SOX-4 protein (X70683), hR-PTPu protein tyrosine phosphatase (X58288), EGR2 (early growth response 2) (J04076), DNA polymerase gamma (U60325), cystathionine beta synthase, alt splice 3 (HG2383-HT4824), CPBP (DNA-binding protein CPBP) (U44975), skeletal muscle C-protein (X66276), HU-K5 (lysophospholipase homolog) (U67963), fibromodulin (U05291), prostatin (L41351), apolipoprotein E (M12529), hEGR1 (early growth response 1) (X52541), DNA polymerase beta (D29013), GOS3 (L49169), ANK-3 (Ankyrin G) (U13616), Gap junction protein (X04325), Hepsin (X07732), CYP1B1 (dioxin-inducible cytochrome P450 (U03688), T-cell receptor Ti rearranged gamma chain V-J-C (M30894), KIAA00167 (D28589), ornithine decarboxylase (M33764), Tob (D38305), 17-beta-hydroxysteroid dehydrogenase (X87176), homeo box c8 protein (M16938), TRAIL (TNF-related apoptosis inducing ligand (U37518), cellular onco-fos (V01512), ESE-1 (epithelial-specific transcription factor) (U73843), prostate-specific membrane antigen, alternatively spliced (S76978), prostate-specific membrane antigen (M99487), T-cell receptor Ti rearranged gamma chain V-J-C region (M30894), OSF-2os (Osteoblast specific factor 2) (D13666), LDL phospholipase A2 (U24577), MAOA (monoamine oxidase A) (M68840), ALCAM (CD6 ligand) (L38608), UDP-GalNAC:polypeptide N-acetylgalactosaminyl transferase (X92689), NB thymosin beta (D82345), FBP1 (Fructose-1,6 biphosphatase), NMB (X76534), cytochrome c-1 (J04444), ionizing radiation conferring protein (U18321), Myoglobin exon 1 (X00371), Memc (U30999), Clone 23587 sequence (U90914), pyrroline 5-carboxylate synthetase (X94453), ADE2H1 (X53793), (SNX) sorting nexin 1 (U53225), IMPDH2 (inosine monophospate dehydrogenase type II) (L33842), transcription factor E2F-5 (U31556), propionyl CoA carboxylase beta subunit (S67325), 6-pyruvoyl-tetrahydropterin synthase (D17400), ADP/ATP carrier protein (J02683), nucleoside diphosphate kinase Nm23-H2s (HG1153-HT1153), ormithine decarboxylase (M33764), CLCN3 (X78520), c-fos (V01512), PCC (propionyl-CoA carboxylase bea-subunit) (M31169), adenylsuccinate lyase (X65867), Cctg chaperonine (X74801), SIM2 (U80456), liver gap-junction protein (X04325), C-myc (L00058), HLA-DMB (U15085), carcinoma-associated antigen GA733-2 (M93036), homeo box c8 protein (M16938), GST-1 Hs GTP binding protein (X17219), Brain guanine nucleotide binding protein (M17219), spermidine synthase (M34338), NAD-dependent methylene tetrahydrofolate dehydrogenase cyclohydrolase (E.C. 1.5.1.15) (X16396), C8FW phosphoprotein (AJ000480), NBK apoptotic inducer protein (X89986), TK (transketolase) (L12711), MNK1 (AB000409), fatty acid synthase (S80437), tubulin beta (HG4322-HT4592), testican (X73608), Arg protein kinase-binding protein (X95632), DNA polymerase delta (U21090), IP-30 (gamma-interferon-inducible protein) (J03909, Lutheran blood group glycoprotein (X83425), tyrosine phosphatatase 1 non-receptor (HG3187-HT3366), mestatasis-associated mta-1 (U35113), (RPS6KA2) ribosomal protein S6 kinase 2 (L06797), transcription factor mef2 alt. splice 2 (HG4668-HT5083), basic transcription factor 44 kDa (HG3748-HT4018), soluble guanylate cyclase large subunit (X66534), transcription factor ETR10 (M62831), orphan G-protein-coupled receptor (L06797), MHC Class II W52 (HG3576-HT3779), prostasin (L41351), M6 antigen (X64364), Mrp17 (X79865), Ly-GDI (GDP-dissociation inhibitor protein) (L20688), KH type splicing regulatory protein KSRP (U94832), Ia-associated invariant gamma-chain (M13560), HLA-DRB1 (MHC class II beta1) (M33600), transcriptional activator hSNF2b (D26156), USF2 (AD000684), SEP protein (X87904), nested protein (M34677), HOXA9 (class I homeoprotein) (U41813), BRG1 (transcriptional activator) (U29175), KIAA0075 (D38550), eIF3 (translational initiation factor) (U78525), KIAA0113 (D30755), HU-K5 (lysophospholipase homolog) (U67963), ADP/ATP tranlocase (J03592), inducible poly(A)-binding protein (U33818), KIAA0146 (D63480), NET1 (guanine nucleotide regulatory protein) (U02081), KIAA0162 (D79984), v-ets erythroblastosis virus E26 oncogene like (AI351043), FBJ murine osteosarcroma viral oncogene homolog B (NM_{—}006732.1), ubiquitin D (NM_{—}006398.1), sialyltransferase I (AI743792), RALBP1 associated Eps domain containing 2 (NM_{—}004726.1), chemokine (C-C motif) ligand 19 (U88321.1), transient receptor potential cation channel subfamily M member (NM_{—}01736.1), B cell activation gen(S59049.1), eukaryotic translation initiation factor 4E binding protein 1 (AB044548.1), lymphocyte antigen 75 (NM_{—}002349),
alpha-methylacyl-CoA racemase (NM_{—}014324.1), phosphoprotein regulated by mitogenic pathway (NM_{—}025195.1), RALBP1 associateds Eps domain containing 2 (NM_{—}004726.1), neuropilin (NRP) and tolloid (TLL)-like 2 (NM_{—}018092.1), twist homolog (X99268.1), calcium calmodulin-dependent protein kinase 2 (AA181179), tumor associated calcium signal transducer 1 (NM_{—}002354.1), UDP-N-acetylglucosamine phosphorylase I (S73498.1), epithelial cell transforming sequence 2 oncogene (NM_{—}01898.1), myosin VI (U90236.2), LIM protein (NM_{—}006457.1), claudin 8 (AL049977.1), phosphoprotein regulated by mitogenic pathway (NM_{—}025195.1), thymosin beta (NM_{—}021992.1), TNF (ligand) superfamily (U57059.1), unknown protein (AV715767), activated leucocyte cell adhesion molecule (NM_{—}001627.1), chaperonin containing TCP1 (NM_{—}001762.1), phosphoribosylaminoimidazole carboxylase (AA902652), protein (NM23A) (NM_{—}000269.1); and
identifying the test sample as cancerous when expression of at least one of the first group of RNA transcripts or translation products is found to be lower in the test sample than in the control sample, and expression of at least one of the second group of transcripts or translation products is found to be higher in the test sample than in the control sample.

2. The method of claim 1 further comprising the step of determining the level of expression of RNA transcripts using an array of nucleic acid molecules.

3. The method of claim 1 further comprising the step of comparing the level of expression of at least one RNA transcript in the test sample to the level of expression of said transcript in the control sample.

4. The method of claim 1 further comprising the step of comparing transcripts or translation products of at least two of the genes of the first group.

5. The method of claim 1 further comprising the step of comparing transcripts or translation products of at least two of the genes of the second group.

6. The method of claim 1 further comprising the step of comparing transcripts or translation products of at least five of the genes of the first group.

7. The method of claim 1 further comprising the step of comparing transcripts or translation products of at least five of the genes of the second group.

8. The method of claim 1 further comprising the step of comparing transcripts or translation products of at least ten of the genes of the first group.

9. The method of claim 1 further comprising the step of comparing transcripts or translation products of at least ten of the genes of the second group.

10. The method of claim 1 further comprising the step of comparing transcripts or translation products of at least twenty of the genes of the first group.

11. The method of claim 1 further comprising the step of comparing transcripts or translation products of at least twenty of the genes of the second group.

12. The method of claim 1 further comprising the step of comparing transcripts or translation products of at least thirty of the genes of the first group.

13. The method of claim 1 further comprising the step of determining the expression level of maspin (U04313) transcript or its translation product.

14. The method of claim 1 further comprising the step of determining the expression level of hepsin (X07732) transcript or its translation product.

15. The method of claim 1 further comprising the step of comparing transcripts or translation products of at least two of the genes in each group of RNA transcripts or translation products.

16. The method of claim 1 further comprising the step of comparing transcripts or translation products of at least five of the genes in each group of transcripts or translation products.

17. The method of claim 1 further comprising the step of comparing transcripts or translation products of at least ten of the genes in each group of transcripts or translation products.

18. The method of claim 1 further comprising the step of comparing transcripts or translation products of at least twenty of the genes in each group of transcripts or translation products.

19. The method of claim 1 further comprising the step of comparing at least thirty of the transcripts or translation products in the fast group and twenty of the transcripts or translation products in the second group.

20. The method of claim 1 further comprising the step of comparing at least forty of the transcripts or translation products in the first group and twenty of the transcripts or translation products in the second group.

21. The method of claim 1 wherein the at least one RNA transcript or its translation product of the first group of RNA transcripts or translation products comprises the transcript of the gene maspin (U04313).

22. The method of claim 1 wherein the at least one RNA transcript or its translation product of the second group of RNA transcripts comprises the transcript of the gene hepsin (X07732).

23. The method of claim 1 wherein the test sample comprises Gleason grade 4/5 prostate carcinoma cells.

24. The method of claim 1 wherein the nonmalignant prostate tissue is benign prostate hyperplasia tissue.

25. The method of claim 1 further comprising the step of identifying the test sample as Gleason grade 4/5 prostate carcinoma.
